(19) United States
(12) Patent Application Publication
(10) Pub. No.: US 2008/0102039 A1
(43) Pub. Date: May 1, 2008

(54) Betamethasone spray

(76) Inventor: Stephen Tickle, Appleton (GB)

Correspondence Address: DANIEL B. SCHEIN, PH.D., ESQ., INC., P. O. BOX 68128, Virginia Beach, VA, 23471, US

(21) Appl. No.: 11/657,236

(22) Filed: Jan. 24, 2007

(30) Foreign Application Priority Data
Oct. 28, 2006 (GB) 0621494 4

(51) Int. Cl. A61K 9/12

(52) U.S. Cl. 424/45

(57) ABSTRACT

A spray foaming dosage form comprising betamethasone valerate, dimethyl isosorbide, propylene glycol, non ionic surfactant, sodium dodecyl sulphate, a buffer, optional preservative, optional further excipients, and water.

### FIELD OF THE INVENTION

This invention relates to a spray formulation of betamethasone valerate.

### BACKGROUND OF THE INVENTION

Betamethasone valerate is a synthetic corticosteroid for topical dermatological use. The corticosteroids are primary synthetic steroids that have anti-inflammatory, antipruritic and vasoconstrictive properties. Betamethasone valerate has a high degree of glucocorticoid activity and a low degree of mineralocorticoid activity. Betamethasone valerate is practically insoluble in water.

A previously known dosage form comprises an aerosol of betamethasone valerate and as an excipient, ethanol. Aerosol formulations can be used to administer various active substances but they have the disadvantages of relatively high cost of construction of the container and metered dosage valve. Also the propellant may have undesirable environmental properties.

It is an object of the present invention to provide a non-aerosol spray formulation of betamethasone valerate which does not contain ethanol (or that contains insufficient ethanol to suffer the well known adverse effects of ethanol in formulations applied to the skin, e.g., irritation). This and other objects are met in whole or in part by the present invention.

### SUMMARY OF THE INVENTION

According to a first aspect of the present invention a spray foaming dosage form comprises:

betamethasone valerate,

dimethyl isosorbide,

propylene glycol,

optionally, a non-ionic surfactant; and if present, preferably polysorbate,

sodium dodecyl sulphate,

a buffer,

optional preservative,

optional further excipients, and

water.

### FURTHER DETAILS OF THE INVENTION

The amount of betamethasone valerate is about 0.12% w/w although higher or lower amounts may be used as desired, for example from 0.5% to 2.0%.

Percentages and other amounts referred to in the specification are by weight unless indicated otherwise. Percentages and other proportions are selected from any ranges quoted to total 100%.

The amount of dimethyl isosorbide may be 5 to 15%, preferably 8 to 15%, more preferably 8 to 12%, most preferably about 10%.

The amount of propylene glycol is preferably 5 to 20%, more preferably 10 to 20%, most preferably about 15%. A non-ionic surfactant is preferred in order to reduce irritation to patients having sensitive or compromised skin.

A preferred non-ionic surfactant is polysorbate, preferably polysorbate 80. An amount of 1% to 10% may be used, preferably about 4.0%.

Sodium dodecyl sulphate is used as a foaming agent. An amount of 0.5 to 2.5%, more preferably 0.5 to 1.3%, most preferably about 0.8% may be used.

A buffer is used to produce a foaming formulation having a pH from about 4.5 to about 5.0. A citrate buffer is preferred, for example comprising trisodium citrate dihydrate and anhydrous citric acid. An amount of 0.163% trisodium citrate dihydrate and 0.085% of anhydrous citric acid is preferred.

Any suitable preservative is employed, for example imidazolidinyl urea. A preferred amount of about 0.3% may be employed.

In view of the low solubility of betamethasone valerate (also referred to as “BMV”) in water, dimethyl isosorbide is used as a solvent in conjunction with propylene glycol as a co-solvent in order to prevent precipitation of the active upon storage at low temperatures.

The invention is further described by means of example but not in any limitative sense.

### EXAMPLE 1

### Formulation and Method:

TABLE 1
Formulation 1

| Excipient | % w/w |
|---|---|
| Betamethasone Valerate | 0.12 |
| Dimethyl Isosorbide | 5.0 |
| Propylene Glycol | 10.0 |
| Polysorbate 80 | 4.0 |
| Imidazolidinyl Urea | 0.3 |
| Sodium Dodecyl Sulphate | 0.8 |
| Trisodium Citrate Dihydrate | 0.163 |
| Anhydrous Citric Acid | 0.085 |
| Purified Water | To 100 |

1) Into vessel 1 was added the dimethyl isosorbide to which was slowly sprinkled the BMV. This was left mixing until visually dissolved.
2) To vessel 1 was added the propylene glycol with stirring until homogenous.
3) In vessel 2 was added the required amount of water and polysorbate 80. This was stirred until dissolved and homogenous.
4) The 3Na.citrate.2H₂O, anhyd. citric acid, imidazolidinyl urea and SDS were then added to vessel 2 and stirred until completely dissolved.
5) The contents of vessel 2 were then poured into vessel 1 and stirred until homogenous.

The BMV successfully dissolved into the dimethyl isosorbide without any difficulties. When the Active Pharmaceutical Ingredient (hereinafter “API”) phase was then poured into the aqueous phase there was no visual precipitation of the BMV when observed over the next few days. A 1.1 kg batch of the 0.12% w/w Betamethasone valerate foam formulation above was then made up for a 3-month accelerated stability study using the same method: (Table 2)

TABLE 2
Formulation 1 Stability Batch

| Excipient | % w/w | In 1.1 kg (g) | Actual Used (g) |
|---|---|---|---|
| Betamethasone Valerate | 0.12 | 1.32 | 1.322 |
| Dimethyl Isosorbide | 5.0 | 55.0 | 55.003 |
| Propylene Glycol | 10.0 | 110.0 | 110.003 |
| Polysorbate 80 | 4.0 | 44.0 | 44.014 |
| Imidazolidinyl Urea | 0.3 | 3.3 | 3.301 |
| Sodium Dodecyl Sulphate | 0.8 | 8.8 | 8.802 |
| Trisodium Citrate Dihydrate | 0.163 | 1.793 | 1.793 |
| Anhydrous Citric Acid | 0.085 | 0.935 | 0.935 |
| Purified Water | To 100 | To 1100 | To 1100 |

After 3 months the samples showed no sign of API precipitation or crystallisation when viewed under the light microscope.

### EXAMPLE 2

### Investigation:

500 g of the 0.12% betamethasone valerate foam solution was made up in accordance with Table 3 below:

TABLE 3
500 g 0.12% Betamethasone Valerate Formulation

| | | | | Actual |
|---|---|---|---|---|
| | Ingredient | % w/w | In 500 g (g) | Used (g) |
| Active Phase | Betamethasone | 0.12 | 0.6 | 0.600 |
| | Valerate | | | |
| | Dimethyl Isosorbide | 5.0 | 25.0 | 25.009 |
| | Propylene Glycol | 10.0 | 50.0 | 50.008 |
| Aqueous | Polysorbate 80 | 4.0 | 20.0 | 20.003 |
| Phase | Sodium Dodecyl | 0.8 | 4.0 | 4.007 |
| | Sulfate | | | |
| | Imadizonyl Urea | 0.3 | 1.5 | 1.500 |
| | Trisodium Citrate | 0.163 | 0.815 | 0.815 |
| | Dihydrate | | | |
| | Anhydrous Citric Acid | 0.085 | 0.425 | 0.425 |
| | Purified Water | To 100 | To 500 | To 500 |

Note:
This formulation was used to make up 4 different samples in the following experiments. This was not made up as one batch.

All glassware in the experiment was rinsed out once with purified water directly from the Millipore Milli-U10 water purification dispenser. They were then rinsed out 3 times with filtered purified water and dried in the Gallenkamp oven and then left to cool to room temperature.

### 1. Dissolution of Betamethasone Valerate into Dimethyl Isosorbide:

The DMI phase was made up for a total solution volume of 500 g so 0.600 g (actual weight) of betamethasone valerate was dissolved in 25.009 g of Arlasolve DMI. The rate of addition was 0.600 g added over 30 secs that is at a rate of 0.02 g/sec. The solution was then mixed at speed setting 1 on the Bibby magnetic stirrer. 1 drop of the solution was then taken every 5 mins. and examined under the Leica DM LB microscope with a 5× lens and L plan 10× eyepiece. A 100×1 mm² graticule was placed over the sample. The temperatures before and after addition of DMI were also recorded.

### Results:

Temperature of DMI=19.3° C.

Temperature after addition of BMV=20.4° C.

There was a temperature increase of 1.1° C.

TABLE 4
Observations of BMV/DMI solution

| Time (min.) | Observations Under Microscope |
|---|---|
| 5 | 3 crystals observed. |
| 10 | 1 crystal observed. |
| 15 | No crystals observed. |
| 20 | 3 crystals observed. |
| 25 | 1 crystal observed. |
| 30 | No crystals observed. |
| 35 | No crystals observed. |

It was observed that the betamethasone valerate takes at least 30 minutes to fully dissolve into the dimethyl isosorbide.

### 2. Addition of Propylene Glycol to the BMV/DMI Phase:

Whilst stirring the BMV/DMI solution at speed 1 on the Bibby magnetic stirrer, the propylene glycol (50.008 g) was poured in over approx. 12 secs, at a rate of 4.17 g/sec. 1 drop of the solution was then taken every 5 mins. and examined under a Leica DM LB microscope with a 5× lens and L plan 10× eyepiece. A 100×1 mm² graticule was placed over the sample. The temperatures before and after addition of propylene glycol were also recorded.

### Results:

Temperature of BMV/DMI solution=20.4° C.

Temperature after addition of BMV=18.0° C.

There was a temperature decrease of 2.4° C.

TABLE 5
Observations of BMV/DMI solution with propylene glycol

| Time (min.) | Observations Under Microscope |
|---|---|
| 5 | No crystals observed. |
| 10 | No crystals observed. |
| 15 | No crystals observed. |

The addition of propylene glycol clearly was observed to have no effect on precipitating the betamethasone valerate out into solution.

### 3. Effect of Additions of Aqueous Phase to the Active Phase/Active Phase to Aqueous Phase:

The aqueous phase was made up to the amount required in a 500 g batch of the foam solution (see Table 1 for amounts used). To the required amount of water was added the polysorbate 80, trisodium citrate dihydrate, anhydrous citric acid, imadizonyl urea and sodium dodecyl sulfate. These were completely dissolved into solution individually with the required speed setting of 2 on the Bibby magnetic stirrer. Total preparation time was 16 mins, with each addition taking no more than 5 secs to add (excluding the water phase). This solution was split into 4 aliquots for the experiments.

a. Aliquot 1: Add the Active Phase to the Aqueous Phase:

15.120 g of the active phase was added to 84.880 g of the aqueous phase over 10 secs (Total vol.=100.000 g), at a rate of 1.51 g/sec. The solution was stirred at speed setting 1 on the Bibby magnetic stirrer. 1 drop of the solution was then taken every 5 mins. and examined under the Leica DM LB microscope with a 5× lens and L plan 10× eyepiece. A 100×1 mm² graticule was placed over the sample. The temperatures before and after addition of the active phase were also recorded.

### Results:

Temperature of aqueous phase=21.4° C.

Temperature after addition of active phase=25.9° C.

There was a temperature increase of 4.5° C.—Slight exothermic reaction.

TABLE 6
Observations of Aliquot 1

| Time (mins.) | Observations Under Microscope |
|---|---|
| 5 | No crystals observed. |
| 10 | No crystals observed. |
| 15 | No crystals observed. |

No effect on precipitation of betamethasone valerate was observed.

b. Aliquot 2: Add the Active Phase to the Aqueous Phase at 20° C.:

15.121 g of the active phase was added to 84.880 g of the aqueous phase over 10 secs (Total vol.=100.001 g, at a rate of 1.51 g/sec. The solution was stirred at speed setting 1 on the Bibby magnetic stirrer. 1 drop of the solution was then taken every 5 mins. and examined under the Leica DM LB microscope with a 5× lens and L plan 10× eyepiece. A 100×1 mm² graticule was placed over the sample. The temperatures before and after addition of the active phase were also recorded.

### Results:

Temperature of aqueous phase=20.0° C.

Temperature after addition of active phase=24.4° C.

A Temperature increase of 4.4° C. was observed, that is a slight exothermic reaction.

TABLE 7
Observations of Aliquot 2

| Time (mins.) | Observations Under Microscope |
|---|---|
| 5 | No crystals observed. |
| 10 | No crystals observed. |
| 15 | No crystals observed. |

There was no effect on precipitation of betamethasone valerate.

c. Aliquot 3: Add the Aqueous Phase to the Active Phase:

84.882 g of the aqueous phase were added to 15.129 g of the active phase over 10 secs (Total vol.=100.011 g). The rate of addition was 8.49 g/sec. The solution was stirred at speed setting 1 on the Bibby magnetic stirrer. 1 drop of the solution was then taken every 5 minutes. and examined under the Leica DM LB microscope with a 5× lens and L plan 10× eyepiece. A 100×1 mm² graticule was placed over the sample. The temperatures before and after addition of the aqueous phase were also recorded.

### Results:

Temperature of aqueous phase=21.2° C.

Temperature after addition of active phase=25.0° C.

A Temperature increase of 3.8° C. was observed, that is a slight exothermic reaction.

TABLE 8
Observations of Aliquot 3

| Time (mins.) | Observations Under Microscope |
|---|---|
| 5 | No crystals observed. |
| 10 | No crystals observed. |
| 15 | No crystals observed. |

There was no effect on precipitation of betamethasone valerate.

d. Aliquot 4: Add the Aqueous Phase at 20° C. to the Active Phase:

84.882 g of the aqueous phase were added to 15.120 g of the active phase over 10 secs (Total vol.=100.002 g), at a rate of 8.49 g/sec. The solution was stirred at speed setting 1 on the Bibby magnetic stirrer. 1 drop of the solution was then taken every 5 mins. and examined under the Leica DM LB microscope with a 5× lens and L plan 10× eyepiece. A 100×1 mm² graticule was placed over the sample. The temperatures before and after addition of the aqueous phase were also recorded.

### Results:

Temperature of aqueous phase=20.1° C.

Temperature after addition of active phase=23.7° C.

There was a temperature increase of 3.6° C.—Slight exothermic reaction.

TABLE 9
Observations of Aliquot 4

| Time (mins.) | Observations Under Microscope |
|---|---|
| 5 | No crystals observed. |
| 10 | No crystals observed. |
| 15 | No crystals observed. |

There was no effect on precipitation of betamethasone valerate.

In addition to the aliquots 1-4 produced 1 drop of the aqueous phase was examined under the Leica DM LB microscope with a 5× lens and L plan 10× eyepiece. A 100×1 mm² graticule was placed over the sample. The microscope revealed no crystals to be present.

I claim:

1. A spray foaming dosage form comprising:
betamethasone valerate,
dimethyl isosorbide,
propylene glycol,
sodium dodecyl sulphate,
a buffer; and
water.

2. A spray foaming dosage form according to claim 1 further comprising a preservative.

3. A spray foaming dosage form according to claim 1 further comprising at least one excipient.

4. A spray foaming dosage form according to claim 1 wherein the amount of said betamethasone valerate is about 0.12%.

5. A spray foaming dosage form according to claim 1 wherein the amount of said dimethyl isosorbide is 5 to 15%.

6. A spray foaming dosage form according to claim 5 wherein the amount of said dimethyl isosorbide is 8 to 15%.

7. A spray foaming dosage form according to claim 6 wherein the amount of said dimethyl isosorbide is 8 to 12%.

8. A spray foaming dosage form according to claim 7 wherein the amount of said dimethyl isosorbide is about 10%.

9. A spray foaming dosage form according to claim 1 wherein the amount of said propylene glycol is 5% to 20%.

10. A spray foaming dosage form according to claim 9 wherein the amount of said propylene glycol is 10% to 20%.

11. A spray foaming dosage form according to claim 10 wherein the amount of said propylene glycol is about 15%.

12. A spray foaming dosage form according to claim 1 further comprising a non-ionic surfactant.

13. A spray foaming dosage form according to claim 12 wherein said non-ionic surfactant comprises polysorbate.

14. A spray foaming dosage form according to claim 13 wherein said non-ionic surfactant comprises polysorbate 80.

15. A spray foaming dosage form according to claim 14 wherein the amount of said polysorbate 80 is from 2 to 6%

16. A spray foaming dosage form according to claim 15 wherein the amount of said polysorbate 80 is about 4.0%.

17. A spray foaming dosage form according to claim 1 wherein the amount of said sodium dodecyl sulphate is 0.5% to 2.5%.

18. A spray foaming dosage form according to claim 17 wherein the amount of said sodium dodecyl sulphate is from 0.5% to 1.3%.

19. A spray foaming dosage form according to claim 18 wherein the amount of said sodium dodecyl sulphate is about 0.8%.

20. A spray foaming dosage form according to claim 1 wherein the formulation has a pH of about 4.5 to about 5%.

21. A spray foaming dosage form according to claim 2 wherein said preservative comprises imidazolidinyl urea.

22. A spray foaming dosage form according to claim 21 wherein the amount of said imidazolidinyl urea is about 0.3%.
